(19) United States
(12) Patent Application Publication
(10) Pub. No.: US 2020/0155459 A1
(43) Pub. Date: May 21, 2020

(54) TABLET AND METHOD FOR MANUFACTURING THE SAME

(71) Applicant: Murata Manufacturing Co., Ltd., Nagaokakyo-shi (JP)

(72) Inventors: Takahiro HIRAO, Nagaokakyo-shi (JP); Fumio NARUSE, Nagaokakyo-shi (JP); Hiroo NOGAMI, Nagaokakyo-shi (JP); Tsuyoshi NAKAGAWA, Nagaokakyo-shi (JP)

(21) Appl. No.: 16/687,972

(22) Filed: Nov. 19, 2019

(30) Foreign Application Priority Data
Nov. 21, 2018 (JP) 2018-218049

(51) Int. Cl. A61K 9/24; A61K 9/28; A61J 3/10

(52) U.S. Cl. CPC A61K 9/209; A61J 3/10; A61K 9/2893

(57) ABSTRACT

A tablet includes a base body and multiple medicine layers stacked inside the base body with a gap between each other in a first direction and including a drug active ingredient. The base body includes multiple inner layers located between adjacent medicine layers in the first direction. The inner layers have a solubility lower than that of the medicine layers.

### CROSS REFERENCE TO RELATED APPLICATIONS

This application claims the benefit of priority to Japanese Patent Application No. 2018-218049 filed on Nov. 21, 2018. The entire contents of this application are hereby incorporated herein by reference.

### BACKGROUND OF THE INVENTION

### 1. Field of the Invention

The present invention relates to tablets and a method for manufacturing the tablets.

### 2. Description of the Related Art

Hitherto, tablets described in Japanese Unexamined Patent Application Publication No. 2005-507925 are known. In the tablets, multiple structure bodies having different solubilities are constituted into a nested state. Specifically, multiple structure bodies are disposed coaxially so as to overlap in the radical direction.

It has been found that when the above-described tablets are actually manufactured and used, the tablets have the following problems.

That is, since multiple structure bodies are in a nested state, the tablet has a complicated structure, and manufacturing of the tablet is difficult. As this manufacturing method, known as compression tableting, in which a powder is compressed to produce a structure body, another structure body is subsequently produced on the outside on the formerly produced structure body with the formerly produced structure body at the center, and this process is repeated to manufacture a tablet. It is thus difficult to adjust the positions of an inner structure body and an outer structure body, and it takes time to manufacture a nested structure.

In addition, since multiple structure bodies are in a nested state, adjustment of the positions of adjacent structure bodies requires simultaneous adjustment of both the radial direction and the axial direction, and it is difficult to form multiple layers.

### SUMMARY OF THE INVENTION

Preferred embodiments of the present invention provide tablets that each have a simple multilayer structure and are able to be easily manufactured and methods for manufacturing the tablets.

A tablet according to a preferred embodiment of the present invention includes a base body; and multiple medicine layers stacked inside the base body with a gap between each other in a first direction and including a drug active ingredient, wherein the base body includes multiple inner layers located between adjacent medicine layers in the first direction; and the inner layers have a solubility lower than that of the medicine layers.

In a tablet according to a preferred embodiment of the present invention, since multiple medicine layers are stacked in a first direction, the tablet can have a simple multilayer structure and can be easily manufactured. In addition, since the multiple medicine layers are stacked in a first direction, the positions of adjacent medicine layers can be adjusted by adjusting the first direction only, and multiple layers can be easily formed.

In addition, when the starting of dissolution of the tablet from both sides in the first direction is observed, the medicine layers and the inner layers are alternately dissolved. Consequently, the timing of dissolution of adjacent medicine layers can be adjusted by the inner layer between the adjacent medicine layers, and maintenance of a drug blood level can be adjusted.

In a tablet according to a preferred embodiment of the present invention, the base body includes outermost layers located on the outer sides in the first direction of the medicine layers that are located at both ends in the first direction.

According to the present preferred embodiment, when the starting of dissolution of the tablet from both sides in the first direction is observed, the time at which the medicine layer starts to dissolve (i.e., start of working of the drug) can be determined by only adjusting the thickness of the outermost layer of the base body.

In addition, in a tablet according to a preferred embodiment of the present invention, the outer circumferential edges of the opposing upper and lower surfaces of the base body in the first direction include steps.

According to the present preferred embodiment, since the outer circumferential edges of the upper and lower surfaces of the base body include steps, the tablet can have a structure with chamfered corners. Consequently, chipping of the corners of the tablet can be reduced or prevented, the tablet can be easily held at the corners to be prevented from being dropped, and the tablet is hardly caught in the throat when the tablet is swallowed.

In addition, in a tablet according to a preferred embodiment of the present invention, the thicknesses of the medicine layers located at both ends in the first direction are larger than those of other medicine layers.

According to the present preferred embodiment, since the thicknesses of the medicine layers at both ends are larger than those of other medicine layers, the amounts of the medicine layers at both ends can be higher than those of other medicine layers. Consequently, when the starting of dissolution of the tablet from both sides in the first direction is observed, the large amounts of the medicine layers at both ends are first dissolved, and the drug can start working sooner.

In formation of steps at the outer circumferential edges of the upper and lower surfaces of the base body, when sheet layers are stacked in the first direction to define a stacked product, if medicine layers are provided only at the central portion of the base body, the base body at the outer circumferential edges not provided with medicine layers becomes thin, and as a result, steps can be provided at the outer circumferential edges of the upper and lower surfaces of the base body. In addition, the difference in level of the steps can be increased by providing thick medicine layers on sheet layers defining the base body and then stacking the sheet layers in a first direction to define a stacked product.

In a tablet according to a preferred embodiment of the present invention, the base body includes multiple inner layers located between adjacent medicine layers in the first direction; outermost layers located on the outer sides in the first direction of the medicine layers that are located at both ends in the first direction; and a side gap portion located on the outer side in a direction orthogonal or substantially orthogonal to the first direction than the medicine layers, wherein a sum of thicknesses of the multiple inner layers and thicknesses of the outermost layers at both ends is not larger than twice the width of the side gap portion.

Here, the thickness of the inner layer and the thickness of the outermost layer are dimensions in the first direction. The width of the side gap portion is a dimension in a direction orthogonal or substantially orthogonal to the first direction and refers to the shortest distance from the surface of the base body to the medicine layer in a direction orthogonal or substantially orthogonal to the first direction in the side gap portion. For example, when the base body has a rectangular or substantially rectangular parallelepiped shape, the width is a dimension in the L direction or the W direction, and when the base body has a cylindrical or substantially cylindrical shape, the width is a dimension in the radial direction.

According to the present preferred embodiment, since the sum of the thicknesses of multiple inner layers and the thicknesses of the outermost layers at both ends is not larger than twice the width of the side gap portion, the side gap portion can remain until all the medicine layers are dissolved, and the effective time of the drug can be controlled.

In a tablet according to a preferred embodiment of the present invention, the inner layers, the outermost layers, and the side gap portion have the same or substantially the same solubility.

According to the present preferred embodiment, since the inner layer, the outermost layer, and the side gap portion can be dissolved at the same speed, the side gap portion can certainly remain until all the medicine layers are dissolved by controlling only the thickness of each layer.

In addition, in a tablet according to a preferred embodiment of the present invention, the base body includes multiple inner layers located between adjacent medicine layers in the first direction; outermost layers located on the outer sides in the first direction than the medicine layers that are located at both ends in the first direction; and a side gap portion located on the outer side in a direction orthogonal or substantially orthogonal to the first direction than the medicine layers, wherein the solubility of the side gap portion is lower than those of the inner layers and the outermost layers, and the solubilities of the inner layers and the outermost layers are lower than that of the medicine layers.

According to the present preferred embodiment, the side gap portion is less soluble than the inner layers and the outermost layers, and the inner layers and the outermost layers are less soluble than the medicine layers. Consequently, the side gap portion remains until all the medicine layers are dissolved.

In addition, in a tablet according to a preferred embodiment of the present invention, the solubility of the outermost layers is lower than that of the inner layers.

According to the present preferred embodiment, since the outermost layers are less soluble than the inner layers, the time at which the medicine layers start to dissolve is able to be delayed.

In addition, in a tablet according to a preferred embodiment of the present invention, the solubility of the outermost layers is higher than that of the inner layers.

According to the present preferred embodiment, since the outermost layers are easily dissolved compared to the inner layers, the time at which the medicine layers start to dissolve can be advanced.

In addition, in a tablet according to a preferred embodiment of the present invention, the medicine layer includes a first effective layer including a first drug active ingredient; a second effective layer including a second drug active ingredient different from the first drug active ingredient; and an intermediate layer located between the first effective layer and the second effective layer and connecting the first effective layer and the second effective layer.

According to the present preferred embodiment, the first effective layer and the second effective layer are able to be prevented from being mixed with each other by dissolving, and the first effective layer and the second effective layer can be prevented from being mixed and reacting with each other before use.

In addition, a method for manufacturing tablets according to a preferred embodiment of the present invention includes a step of providing medicine layers each including a drug active ingredient on respective multiple sheet layers defining a base body; a step of stacking the multiple sheet layers provided with the medicine layers in a first direction to define a stacked product; and a step of pressing the stacked product in the first direction.

According to the present preferred embodiment, multiple medicine layers are able to be stacked in a first direction, and a simple multilayer structure is able to be easily manufactured.

According to the tablets and the methods for manufacturing the tablets according to preferred embodiments of the present invention, the tablets are able to have a simple multilayer structure and can be easily manufactured.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a perspective view illustrating a tablet according to a first preferred embodiment of the present invention.

FIG. 2 is an LT cross-sectional view of the tablet.

FIG. 3 is a diagram of the effectiveness of a drug.

FIG. 4A is a cross-sectional view of a round tablet according to a Comparative Example.

FIG. 4B is a diagram of the effectiveness of a drug according to the Comparative Example.

FIG. 5A shows a method for manufacturing tablets according to a preferred embodiment of the present invention.

FIG. 5B shows a method for manufacturing tablets according to a preferred embodiment of the present invention.

FIG. 5C shows a method for manufacturing tablets according to a preferred embodiment of the present invention.

FIG. 5D shows a method for manufacturing tablets according to a preferred embodiment of the present invention.

FIG. 5E shows a method for manufacturing tablets according to a preferred embodiment of the present invention.

FIG. 6 is a cross-sectional view illustrating a tablet according to a second preferred embodiment of the present invention.

FIG. 7 is a perspective view illustrating a tablet according to a third preferred embodiment of the present invention.

### DETAILED DESCRIPTION OF THE PREFERRED EMBODIMENTS

Preferred embodiments of the present invention will now be described in detail with reference to examples of preferred embodiments as shown in the drawings.

### First Preferred Embodiment

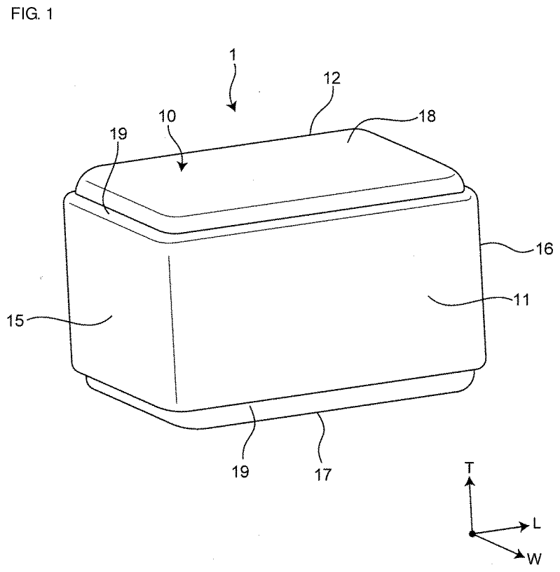

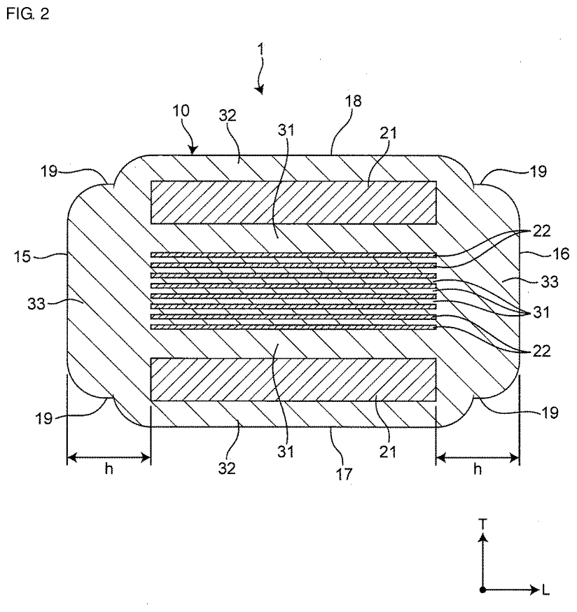

FIG. 1 is a perspective view illustrating a tablet according to a first preferred embodiment of the present invention. FIG. 2 is a cross-sectional view of the tablet. As shown in FIGS. 1 and 2, the tablet 1 includes a base body 10 and multiple medicine layers 21, 22 provided inside the base body 10.

The base body 10 is configured in a rectangular or approximately rectangular solid state. The surface of the base body 10 includes a first end surface 11, a second end surface 12 opposing the first end surface 11, a first side surface 15 extending between the first end surface 11 and the second end surface 12, a second side surface 16 opposing the first side surface 15, a lower surface 17 extending between the first side surface 15 and the second side surface 16, and an upper surface 18 opposing the lower surface 17.

As shown in FIGS. 1 and 2, the L direction is a direction in which the first side surface 15 and the second side surface 16 oppose to each other and is the length direction of the tablet 1. The W direction is a direction in which the first end surface 11 and the second end surface 12 oppose to each other and the width direction of the tablet 1. The T direction (first direction) is a direction in which the lower surface 17 and the upper surface 18 oppose to each other and the thickness direction of the tablet 1. The forward direction of the T direction is the upward direction, and the backward direction of the T direction is the downward direction.

The size of the base body 10 is not limited to the normal tablet size and may preferably be, for example, a size of about 0.6 mm (L dimension)×about 0.3 mm (W dimension) or a size of about 0.4 mm (L dimension)×about 0.2 mm (W dimension) or may be a granular size. The tablet can be smoothly taken into the body by decreasing the size of the tablet.

The multiple medicine layers 21, 22 have a sheet shape and are stacked inside the base body 10 with a gap between each other in a first direction. The multiple medicine layers 21, 22 contain a drug active ingredient. The multiple medicine layers 21, 22 include first medicine layers 21 located at both ends in the T direction and multiple second medicine layers 22 located between the first medicine layers 21 at both ends. In another example, a single second medicine layer 22 may be provided. In such a case, the medicine layers include three layers: two first medicine layers 21 and one second medicine layer 22.

The drug active ingredient may be any active ingredient that is used as solid medicine, and examples thereof include antihyperlipidemic agents, antiulcer agents, antihypertensive agents, antidepressant agents, psychotropic agents, anxiolytic agents, hypnotic sedative agents, antiasthmatic agents, antitussive expectorant agents, antiepileptic agents, agents for dental and oral use, antiallergic agents, antihistamine agents, antibacterial agents, anticancer agents, nourishing and health care agents, antipyretic analgesic antiphlogistic agents, analgesic agents, anti-inflammatory agents, vasoconstrictor agents, coronary vasodilator agents, peripheral vasodilator agents, antidiabetic agents, antimetabolite agents, osteoporosis agents, antirheumatic agents, antispasmodic agents, central nervous system agents, skeletal muscle relaxants, antiplatelet agents, antacids, antiemetic agents, hormone agents, anesthetic agents, alkaloid narcotics, sulfa agents, cholagogues, antibiotics, chemotherapeutic agents, diuretic agents, respiratory stimulants, cardiotonic agents, arrhythmic agents, brain metabolism improving agents, cerebral circulation improving agents, sympathomimetic agents, gastrointestinal agents, gout therapeutic agents, and blood coagulation inhibitors.

The base body 10 includes multiple inner layers 31 located between adjacent medicine layers 21, 22 in the T direction, outermost layers 32 located on the outer sides in the T direction of the medicine layers 21 that are located at both ends in the T direction, and a side gap portion 33 located on the outer side in a direction orthogonal or substantially orthogonal to the T direction of the medicine layers 21, 22. The inner layers 31 and the outermost layers 32 overlap the medicine layers 21, 22 when viewed from the T direction, and the side gap portion 33 does not overlap the medicine layers 21, 22 when viewed from the T direction.

The solubility of the inner layers 31 is preferably lower than that of the medicine layers 21, 22. Here, the term “solubility” refers to the easiness of dissolution of a subject in the body, such as the stomach or intestine. In other words, the term “solubility” refers to a relative comparison of the mass of a subject dissolved in unit time. Low solubility means that the subject is difficult to dissolve in the body. That is, the inner layers 31 are less soluble than the medicine layers 21, 22. Preferably, the inner layers 31, the outermost layers 32, and the side gap portion 33 are made of the same material and have the same or substantially the same solubility.

The components of the inner layers 31, the outermost layers 32, and the side gap portion 33 may be any components other than drug active ingredients, and examples thereof include pharmaceutical additives, such as excipients, disintegrators, moisture-proofing agents, stabilizers, binders, coating agents, antistatic agents, sugar coatings, emulsifiers, softeners, and solvents. In addition, for example, bioceramics, such as apatite, tricalcium phosphate (TCP), aluminum oxide, silicon dioxide, silicon nitride, zirconium phosphate, glass (silica glass, silicate glass, etc.), glass ceramics (crystallized glass, phosphate glass, etc.), and porous glass, can also be used. The content of the pharmaceutical additive can be appropriately determined depending on the type of the additive, the content of the pharmaceutical active ingredient, and the like.

In the tablet 1, since the multiple medicine layers 21, 22 are stacked in the T direction, the tablet can have a simple multilayer structure and can be easily manufactured. In contrast, in the known nested structure, it is required to form medicine layers having a cylindrical or substantially cylindrical shape and to stack the multiple medicine layers in the radial direction, and the manufacturing requires more time.

In the tablet 1, since the multiple medicine layers 21, are stacked in the T direction, the positions of adjacent medicine layers 21, 22 can be adjusted by adjusting the T direction only, and multiple layers can be easily formed. In contrast, in the known nested structure, it is required to simultaneously adjust both the radial direction and the axial direction to adjust the positions of adjacent cylindrical medicine layers, and formation of multiple layers is difficult.

Dissolution of the tablet 1 will now be described.

All surfaces of the base body 10 simultaneously start to dissolve. Here, dissolution of the base body 10 in the T direction is observed. First, the outermost layers 32 on both sides of the base body 10 in the T direction start to dissolve, and when the exposed upper and lower first medicine layers 21 are dissolved, the drug starts to work. Subsequently, the inner layers 31 of the base body 10 interrupt the release of the drug active ingredient. The inner layers 31 are then dissolved, and the exposed second medicine layers 22 are dissolved to start the release of the drug active ingredient.

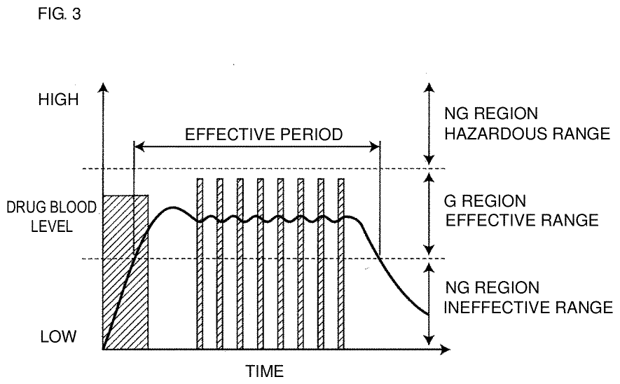

FIG. 3 shows a diagram of the effectiveness of a drug. FIG. 3 shows a relationship between time and blood levels. The solid line shows the relationship between time and blood levels, and the rectangular hatched parts show the amount of the dissolved drug. When the blood level of a drug is too high, the level enters a hazardous range (an NG region). In contrast, when the blood level is too low, the level enters an ineffective range (another NG region). That is, a certain range where the level is neither too high nor too low is the effective range (G region) where the effect safely occurs.

As shown by the solid line in FIG. 3, first, the first medicine layers 21 start to dissolve to gradually increase the blood level, and the peak of the blood level occurs after the first medicine layers 21 are completely dissolved. Subsequently, the inner layers 31 are dissolved, and the second medicine layers 22 are then dissolved. Consequently, the blood level is maintained within the G region. By repeating this process, the second medicine layers 22 and the inner layers 31 are alternately dissolved. Accordingly, the drug blood level remains within the G region for a long time, and the drug effective time can be increased. In addition, variation in the drug blood level over time can be reduced.

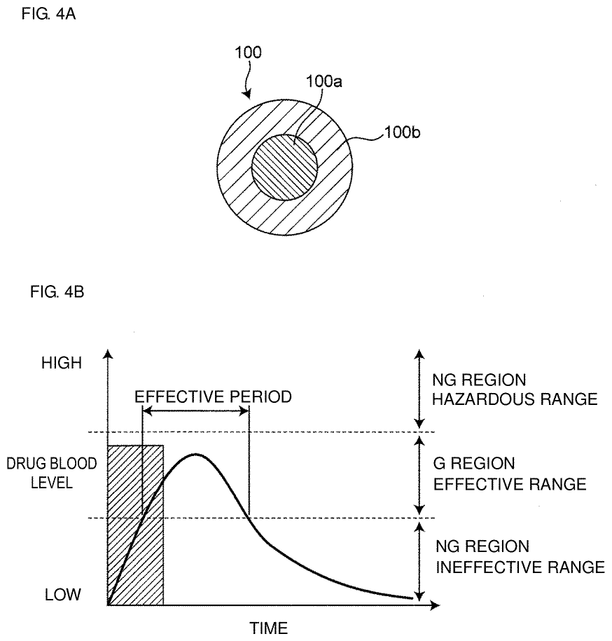

In contrast, the round tablet 100 of a Comparative Example will be described using FIGS. 4A and 4B. As shown in FIG. 4A, the round tablet 100 includes a medicine layer 100a including an active ingredient and located at the center and a coating layer 100b covering the medicine layer 100a. FIG. 4B shows a relationship between time and blood levels in the round tablet 100. The solid line shows the relationship between time and blood levels, and the rectangular hatched part shows the amount of the dissolved drug.

As shown by the solid line in FIG. 4B, after the coating layer 100b is dissolved, the medicine layer 100a starts to dissolve to gradually increase the blood level, and the peak of the blood level occurs after the medicine layer 100a is completely dissolved. Subsequently, since the drug is gradually excreted from the body, the blood level decreases. Accordingly, the drug blood level cannot remain in the G region for an extended time, and the drug effective time is decreased. In addition, the drug blood level varies greatly over time.

According to the tablet 1, when the starting of dissolution of the tablet 1 from both sides in the T direction is observed, the medicine layers 21, 22 and the inner layers 31 are alternately dissolved. Consequently, the timing of dissolution of adjacent medicine layers 21, 22 can be adjusted by the thickness of the inner layer 31 between the adjacent medicine layers 21, 22, and maintenance of the drug blood level can be adjusted.

According to the tablet 1, when the starting of dissolution of the tablet 1 from both sides in the T direction is observed, the time at when the medicine layers 21, 22 start to dissolve (i.e., start of working of the drug) can be determined by only adjusting the thicknesses of the outermost layers 32 of the base body 10.

As shown in FIG. 2, preferably, the sum of the thicknesses of all of the multiple inner layers 31 and the thicknesses of the outermost layers 32 at both ends is not larger than twice the width h of the side gap portion 33. The thickness of the inner layer 31 and the thickness of the outermost layer 32 are dimensions in the T direction. The width h of the side gap portion 33 is a dimension in a direction orthogonal or substantially orthogonal to the T direction and is a dimension in the L direction or the W direction when the base body 10 is a rectangular or substantially rectangular solid.

Here, when the solubility of the side gap portion 33 and the inner layers 31 and the solubility of the outermost layers 32 are sufficiently lower than that of the medicine layers 21, 22, the time for dissolution of the medicine layers 21, 22 is negligible.

Accordingly, although all surfaces of the base body 10 simultaneously start to dissolve, the side gap portion 33 can remain until all the medicine layers 21, 22 are dissolved, and the drug effective time can be adjusted. That is, the medicine layers 21, 22 can elute from the T direction only and are prevented from eluting from a direction orthogonal or substantially orthogonal to the T direction by enabling the side gap portion 33 to remain when the tablet 1 is dissolved.

Preferably, the inner layers 31, the outermost layers 32, and the side gap portion 33 have the same or substantially the same solubility. Consequently, since the inner layers 31, the outermost layers 32, and the side gap portion 33 can be dissolved at the same speed, the side gap portion 33 can remain until all the medicine layers 21, 22 are dissolved by controlling only the thickness of each layer.

As shown in FIG. 2, preferably, the outer circumferential edges of the upper surface 18 and the lower surface 17 of the base body 10 include steps 19. Specifically, the steps 19 are provided on the outer circumferential edge of the side gap portion 33 and are formed by reducing the thickness of the outer circumferential edge in the T direction. In addition, preferably, the surface of the base body 10 has a non-angular shape. Specifically, the portions between the steps 19 and the upper surface 18 or the lower surface 17 of the base body 10 have curved shapes in which the surface extending outward in the T direction gradually extends inward in the direction orthogonal or substantially orthogonal to the T direction. The portions between the steps 19 and the first end surface 11, the second end surface 12, the first side surface 15, or the second side surface 16 have curved shapes in which the surface extending inward in the T direction gradually extends outward in the direction orthogonal or substantially orthogonal to the T direction. The corner between the first side surface 15 and the first end surface 11 adjacent thereto has a curved shape. Furthermore, the corner between the first side surface 15 and the second end surface 12 adjacent thereto, the corner between the second side surface 16 and the first end surface 11 adjacent thereto, and the corner between the second side surface 16 and the first end surface 12 adjacent thereto have curved shapes.

Accordingly, the tablet 1 can have a structure in which the corners are chamfered by providing the steps 19. Consequently, chipping of the corners of the tablets 1 due to collisions or the like between the tablets 1 is able to be reduced.

In addition, since the steps 19 are included at the outer circumferential edges of the upper surface 18 and the lower surface 17 of the base body 10, the portions define and function as catches to prevent dropping during handling in manufacturing. In addition, when a user takes the tablet, dropping can be prevented. It is thus easy to hold the corners of the tablet 1, and dropping of the tablet 1 can be prevented.

In addition, since the steps 19 are provided at the outer circumferential edges of the upper surface 18 and the lower surface 17 of the base body 10, when the tablet 1 is swallowed, the tablet 1 is unlikely to be caught in the throat.

Preferably, the thicknesses of the first medicine layers 21 located at both ends in the T direction are larger than those of the second medicine layers 22. Accordingly, the amounts of the first medicine layers 21 at both ends can be higher than those of the second medicine layers 22. Consequently, when the starting of dissolution of the tablet from both sides in the T direction is observed, the large amounts of the medicine layers 21 at both ends are first dissolved, and the start of working of the drug can be advanced.

In formation of the steps 19 at the outer circumferential edges of the upper surface 18 and the lower surface 17 of the base body 10, sheet layers are stacked in the T direction to form a stacked product. At this time, if the medicine layers 21, 22 are disposed only at a central portion of the base body 10, the base body 10 at the outer circumferential edges where no medicine layers 21, 22 are disposed becomes thin, and as a result, steps 19 can be formed at the outer circumferential edges of the upper surface 18 and the lower surface 17 of the base body 10. In addition, the difference in level of the steps 19 can be increased by providing thick medicine layers 21, 22 on sheet layers defining the base body 10 and then stacking the sheet layers in the T direction to define a stacked product.

Preferably, the solubility of the side gap portion 33 is lower than that of the inner layers 31 and that of the outermost layers 32, and the solubility of the inner layers 31 and the solubility of the outermost layers 32 are lower than that of the medicine layers 21, 22. Accordingly, the side gap portion 33 is less soluble than the inner layers 31 and the outermost layers 32, and the inner layers 31 and the outermost layers 32 are less soluble than the medicine layers 21, 22. Accordingly, the side gap portion 33 can effectively remain until all the medicine layers 21, 22 are dissolved. As long as the side gap portion 33 can remain until all the medicine layers 21, 22 are dissolved, the sum of the thicknesses of all of the inner layers 31 and the thicknesses of the outermost layers 32 at both ends may be larger than twice the width h of the side gap portion 33.

The solubility of the outermost layers 32 is preferably lower than that of the inner layers 31. In such a case, since the outermost layers 32 are less soluble than the inner layers 31, the time at which the medicine layers 21, 22 start to dissolve can be delayed. Alternatively, the solubility of the outermost layers 32 is preferably higher than that of the inner layers 31. In such a case, since the outermost layers 32 are easily dissolved compared to the inner layers 31, the time at which the medicine layers 21, 22 start to dissolve can be advanced.

A method for manufacturing the tablet 1 according to a preferred embodiment of the present invention will now be described.

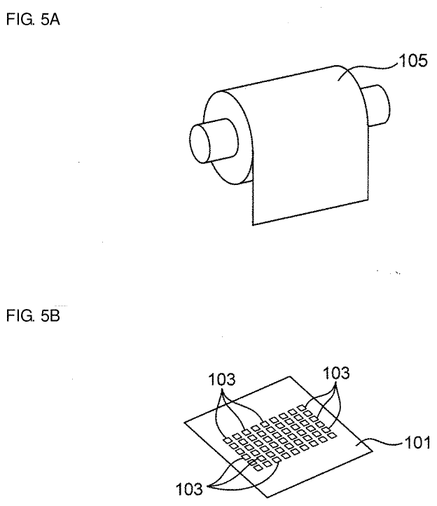

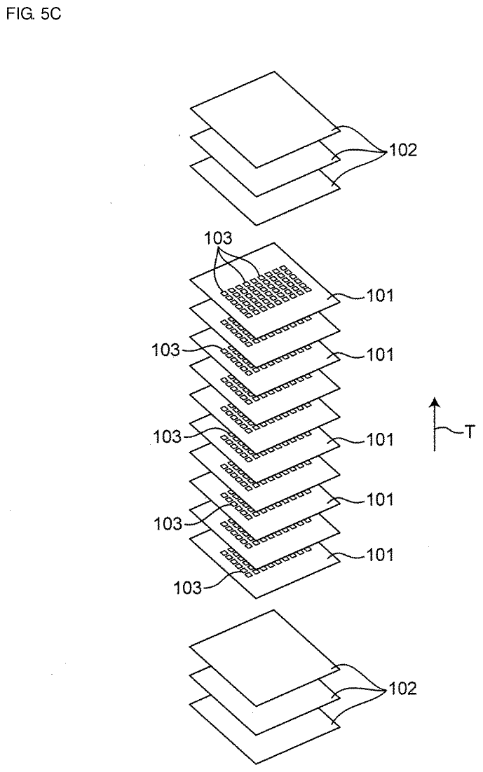

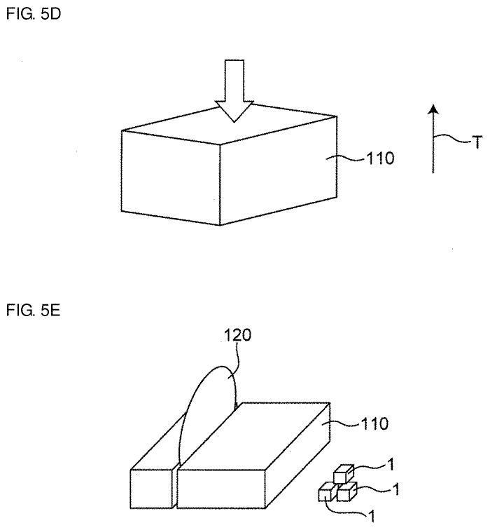

As shown in FIG. 5A, a low-soluble material having a low solubility is produced into a sheet shape to produce a sheet body 105. The low-soluble material is, for example, a material of the above-described inner layers 31. The sheet body 105 is formed by, for example, die coating, doctor blade coating, roll coating, or ink-jet coating. The sheet body 105 is formed at a temperature of, for example, about 20° C. or more and about 80° C. or less. The sheet body 105 is formed at a speed of, for example, about 2 m/min or more and about 200 m/min or less. The sheet body 105 is dried by, for example, one or more of blowing, heating, and reducing the atmospheric pressure. The temperature when the sheet body 105 is heated is, for example, about 45° C. or more and about 100° C. or less.

Subsequently, as shown in FIG. 5B, multiple first sheet layers 101 defining the base body 10 are cut out from the sheet body 105, and medicine layers 103 are provided to each of the multiple first sheet layers 101. The medicine layers 103 are formed by printing paste including a drug active ingredient on the first sheet layer 101. The drug active ingredient is, for example, the drug active ingredient of the above-described medicine layers 21, 22. The medicine layers 103 are formed, for example, by a printing process, such as screen printing, offset printing, intaglio printing, letterpress printing, or ink-jet printing, and the medicine layers 103 may be formed by repeating the same or different printing processes. The temperature for drying the medicine layers 103 is, for example, about 20° C. or more and about 100° C. or less. The medicine layers 103 are dried by, for example, one or more of blowing, heating, and reducing the atmospheric pressure. The temperature when heating is performed is, for example, a constant temperature of about 75° C.

Subsequently, as shown in FIG. 5C, multiple second sheet layers 102 defining the base body 10 are cut out from the sheet body 105, the multiple first sheet layers 101 provided with the medicine layers 103 are stacked in the T direction, and the second sheet layers 102 are stacked on both ends in the T direction to define a stacked product 110 as shown in FIG. 5D. The temperature for pressure bonding of the first sheet layers 101 and the second sheet layers 102 is, for example, about 60° C. or more and about 90° C. or less. The pressure for pressure bonding of the first sheet layers 101 and the second sheet layers 102 is, for example, about 1 MPa or more and about 200 MPa or less at most.

Subsequently, as shown in FIG. 5D, the stacked product 110 is pressed in the T direction. The method for pressing the stacked product 110 is, for example, isotactic pressing, specifically, rigid body pressing using a heated rigid plate, rubber pressing, or isostatic pressing. The temperature for pressing the stacked product 110 is, for example, about 25° C. or more and about 200° C. or less. The pressure for pressing the stacked product 110 is, for example, about 1 MPa or more and about 200 MPa or less at most.

When the stacked product 110 is pressed, as shown in FIG. 2, steps 19 are easily formed at the outer circumferential edges of the upper surface 18 and the lower surface 17 of the base body 10. That is, when the stacked product 110 is pressed, the thickness of the portion (side gap portion 33) where no medicine layers 103 are present on the sheet layers 101 is smaller than the thickness of the portion (outermost layers 32) where the medicine layers 103 are present on the sheet layers 101, and steps 19 are formed. In particular, when the density of the medicine layers 103 is higher than that of the sheet layers 101, 102, the steps 19 are notably formed.

Subsequently, as shown in FIG. 5E, the stacked product 110 is cut into a predetermined size with a dicing blade 120 to form tablets 1. The method for cutting into a predetermined size may be, for example, a method using a force-cutting blade, instead of the dicing, or may be punching.

Subsequently, burrs of the tablets 1 are removed to manufacture the tablets 1. When burrs are removed, the steps 19 may be provided at the outer circumferential edges of the upper surface 18 and the lower surface 17 of the base body 10.

The method for manufacturing the tablets 1 of the present preferred embodiment includes a step of providing medicine layers 103 to each of multiple sheet layers 101, a step of stacking the multiple sheet layers 101 provided with the medicine layers 103 in a T direction to constitute a stacked product 110, and a step of pressing the stacked product 110 in the T direction. Accordingly, the multiple medicine layers 103 can be stacked in the T direction, and a simple multilayer structure can be easily manufactured.

### Second Preferred Embodiment

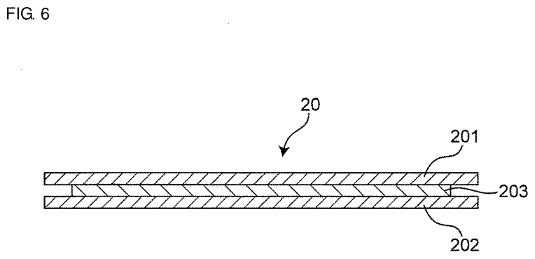

FIG. 6 is a cross-sectional view of a medicine layer 20 of a tablet according to a second preferred embodiment of the present invention. The second preferred embodiment differs from the first preferred embodiment in the configuration of the medicine layer. This different configuration will now be described. Other configurations are the same or substantially the same as those in the first preferred embodiment.

As shown in FIG. 6, in the tablet according to the second preferred embodiment, the medicine layer 20 includes a first effective layer 201 including a first drug active ingredient, a second effective layer 202 including a second drug active ingredient, and an intermediate layer 203 located between the first effective layer 201 and the second effective layer 202 and connecting the first effective layer 201 and the second effective layer 202. That is, the medicine layer 20 of the second preferred embodiment includes three layers.

The first drug active ingredient of the first effective layer 201 and the second drug active ingredient of the second effective layer 202 are preferably different from each other, and examples thereof are the same as those exemplified as the drug active ingredient of the first preferred embodiment.

The component of the intermediate layer 203 may include, for example, a binder (preferably pharmaceutically acceptable binder) other than drug active ingredients. In such a case, the strength, hardness, etc. of the solid medicine can be improved. The binder may be, for example, any binder that has a property of binding powder particles in the presence of water, and binders that are used in a powder lamination molding method can also be used. For example, a water-soluble organic polymer component can be suitably used. More specifically, the water-soluble organic polymer component is, for example, at least one of 1) polyvinylpyrrolidone (PVP), polyvinyl alcohol (PVA), polyethylene glycol (PEG), polyvinyl acetate, polyvinyl butyral, polyacrylic acid, sodium polyacrylate, a copolymer of sodium polyacrylate and maleic acid, and a copolymer of polyvinylpyrrolidone and vinyl acetate, 2) cellulose derivatives (such as methyl cellulose, ethyl cellulose, ethylhydroxymethyl cellulose, carboxymethyl cellulose (CMC), carboxymethyl cellulose sodium, hydroxypropyl cellulose (HPC), and hydroxypropylmethyl cellulose (HPMC)), 3) gum Arabic, Locust gum, gelatin, starch, sucrose, dextrose, fructose, lactose, flour, alginic acid, sodium alginate, etc. and 4) citric acid, succinic acid, etc. Among these polymer components, from the viewpoint of having dispersibility to prevent hardening of powder and characteristics to improve fluidity and enhance the planar spreading properties of powder, it is preferable to use at least one of polyvinylpyrrolidone, carboxymethyl cellulose, hydroxypropyl cellulose, and sodium alginate, for example.

When this medicine layer 20 is dissolved, the first effective layer 201 (or the second effective layer 202), the intermediate layer 203, and the second effective layer 202 (or the first effective layer 201) are dissolved in this order. Accordingly, even if the first effective layer 201 and the second effective layer 202 have properties of reacting with each other (e.g., blending variation) when they are mixed, the first effective layer 201 and the second effective layer 202 can be prevented from being dissolved and mixed with each other before use (before administration), and the first effective layer 201 and the second effective layer 202 can be prevented from being mixed and reacting with each other during storage before use.

In addition, when a drug active ingredient that is generated by reaction between the first effective layer 201 and the second effective layer 202 loses the effectiveness within a short period of time after the generation, there is a need to administer the drug active ingredient immediately after the reaction between the first effective layer 201 and the second effective layer 202. In this configuration, the first effective layer 201 and the second effective layer 202 can be almost simultaneously dissolved by reducing the thickness of the intermediate layer 203. Consequently, it is possible to absorb the drug active ingredient in the body immediately after the generation by reaction between the first effective layer 201 and the second effective layer 202 occurring when administered.

Incidentally, the configuration of the medicine layer 20 of the second preferred embodiment may be applied to at least one medicine layer of the multiple first medicine layers 21 and the multiple second medicine layers 22 in the first preferred embodiment. In addition, the medicine layer 20 may include four or more layers, and the number of the effective layers may be three or more.

### Third Preferred Embodiment

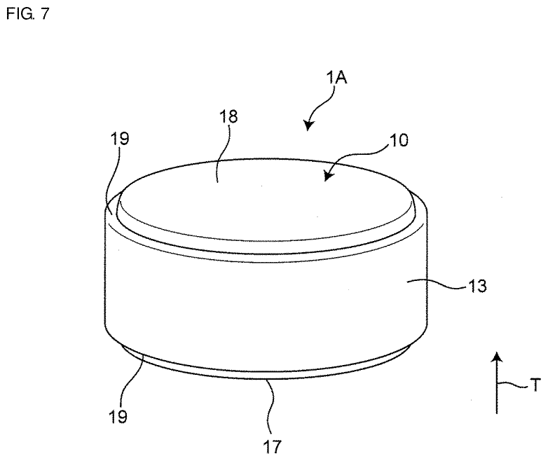

FIG. 7 is a perspective view illustrating a tablet according to a third preferred embodiment of the present invention. The third preferred embodiment differs from the first preferred embodiment in the shape of the base body. This different configuration will now be described. Other configurations are the same or substantially the same as those in the First Preferred Embodiment.

As shown in FIG. 7, in the tablet 1A according to the third preferred embodiment, the base body 10 has a cylindrical or substantially cylindrical shape. The surface of the base body 10 includes a circular lower surface 17, a circular upper surface 18, and a peripheral surface 13 extending between the lower surface 17 and the upper surface 18.

The cross-section along the radial direction of the tablet 1A of the third preferred embodiment is similar to the LT cross-section of the tablet 1 of the first preferred embodiment shown in FIG. 2. That is, the configuration of the tablet 1A of the third preferred embodiment is the same or substantially the same as that of the tablet 1 of the first preferred embodiment excluding the external shape of the base body 10.

Specifically, the multiple medicine layers 21, 22 are stacked inside the base body 10 with a gap between each other in a T direction. Preferably, the outer circumferential edges of the upper surface 18 and the lower surface 17 of the base body 10 include steps 19. Preferably, the sum of the thicknesses of all of the inner layers 31 and the thicknesses of the outermost layers 32 at both ends is not larger than twice the width h of the side gap portion 33. On this occasion, the thickness of the inner layer 31 and the thickness of the outermost layer 32 are dimensions in the T direction. The width h of the side gap portion 33 is a dimension in the radial direction. Descriptions of other configurations are omitted.

The present invention is not limited to the above-described preferred embodiments and can be modified without departing from the gist of the present invention. For example, the respective characteristics of the first to third preferred embodiments may be variously combined.

In the preferred embodiments of the present invention, although the base body has a rectangular or substantially rectangular parallelepiped or cylindrical shape, the shape may be, for example, a rhomboid or a sphere. In such a case, the width of the side gap portion is a dimension in a direction orthogonal or substantially orthogonal to the first direction and refers to the shortest distance from the surface of the base body to the medicine layer in a direction orthogonal to the first direction in the side gap portion.

While preferred embodiments of the invention have been described above, it is to be understood that variations and modifications will be apparent to those skilled in the art without departing from the scope and spirit of the invention. The scope of the invention, therefore, is to be determined solely by the following claims.

What is claimed is:

1. A tablet comprising:
a base body; and
a plurality of medicine layers stacked inside the base body with a gap between each other in a first direction and including a drug active ingredient; wherein
the base body includes a plurality of inner layers located between adjacent medicine layers of the plurality of medicine layers in the first direction; and
the plurality of inner layers have a solubility lower than that of the plurality of medicine layers.

2. The tablet according to claim 1, wherein the base body includes a plurality of outermost layers located on outer sides in the first direction of medicine layers of the plurality of medicine layers that are located at opposite ends in the first direction.

3. The tablet according to claim 1, wherein outer circumferential edges of opposing upper and lower surfaces of the base body in the first direction include steps.

4. The tablet according to claim 1, wherein medicine layers of the plurality of medicine layers located at both ends in the first direction each have a thickness larger than thicknesses of other medicine layers of the plurality of medicine layers.

5. The tablet according to claim 1, wherein the base body further includes:
a plurality of outermost layers located on outer sides in the first direction of medicine layers of the plurality of medicine layers that are located at both ends in the first direction; and
a side gap portion located on an outer side in a direction orthogonal or substantially orthogonal to the first direction of the plurality of medicine layers; wherein
a sum of thicknesses of the plurality of inner layers and thicknesses of the plurality of outermost layers at both ends is not larger than twice a width of the side gap portion.

6. The tablet according to claim 5, wherein the plurality of inner layers, the plurality of outermost layers, and the side gap portion have a same solubility.

7. The tablet according to claim 1, wherein the base body further includes:
a plurality of outermost layers located on outer sides in the first direction of medicine layers of the plurality of medicine layers that are located at both ends in the first direction; and
a side gap portion located on an outer side in a direction orthogonal or substantially orthogonal to the first direction of the plurality of medicine layers; wherein
the side gap portion has a solubility lower than those of the plurality of inner layers and the plurality of outermost layers, and the plurality of inner layers and the plurality of outermost layers have solubilities lower than that of the plurality of medicine layers.

8. The tablet according to claim 7, wherein the plurality of outermost layers have a solubility lower than that of the plurality of inner layers.

9. The tablet according to claim 7, wherein the plurality of outermost layers have a solubility higher than that of the plurality of inner layers.

10. The tablet according to claim 1, wherein the plurality of medicine layers each include:
a first effective layer including a first drug active ingredient;
a second effective layer including a second drug active ingredient different from the first drug active ingredient; and
an intermediate layer located between the first effective layer and the second effective layer and connecting the first effective layer and the second effective layer.

11. The tablet according to claim 1, wherein the base body has a rectangular or substantially rectangular shape.

12. The tablet according to claim 1, wherein the base body has a cylindrical or substantially cylindrical shape.

13. A method for manufacturing a tablet, comprising:
providing a plurality of medicine layers including a drug active ingredient to each of a plurality of sheet layers defining a base body;
stacking the plurality of sheet layers provided with the plurality of medicine layers in a first direction to define a stacked product; and
pressing the stacked product in the first direction.

14. The method for manufacturing a tablet according to claim 13, wherein the plurality of sheet layers have a solubility lower than that of the plurality of medicine layers.

15. The method for manufacturing a tablet according to claim 13, wherein the plurality of medicine layers are formed on the plurality of sheet layers by printing paste including the drug active ingredient of the plurality of sheet layers.

16. The method for manufacturing a tablet according to claim 13, further comprising stacking a plurality of second sheet layers on both side of the plurality of sheet layers in the first direction.

17. The method for manufacturing a tablet according to claim 13, wherein outer circumferential edges of opposing upper and lower surfaces in the first direction of the stacked product after pressing include steps.

18. The method for manufacturing a tablet according to claim 16, wherein the plurality of second sheet layers have a solubility lower than that of the plurality of sheet layers.

19. The method for manufacturing a tablet according to claim 16, wherein the plurality of second sheet layers have a solubility higher than that of the plurality of sheet layers.

20. The method for manufacturing a tablet according to claim 13, wherein the plurality of medicine layers each include:
a first effective layer including a first drug active ingredient;
a second effective layer including a second drug active ingredient different from the first drug active ingredient; and
an intermediate layer located between the first effective layer and the second effective layer and connecting the first effective layer and the second effective layer.
